(19) United States
(12) Patent Application Publication
(10) Pub. No.: US 2010/0168220 A1
(43) Pub. Date: Jul. 1, 2010

(54) PLANT EXTRACT COMPOSITION FOR TREATING HEPATITIS C

(75) Inventors: Lain-Tze Lee, Hsinchu City (TW); Shau-Feng Chang, Hsinchu City (TW); Cheng Lin, Fengyuan City (TW); Shao Chan Yin, Tainan City (TW); Shu-Jiau Chiou, Hsinchu City (TW)

Correspondence Address: PAI PATENT & TRADEMARK LAW FIRM, 1001 FOURTH AVENUE, SUITE 3200, SEATTLE, WA, 98154, US

(73) Assignee: INDUSTRIAL TECHNOLOGY RESEARCH INSTITUTE, Hsinchu County (TW)

(21) Appl. No.: 12/647,496

(22) Filed: Dec. 27, 2009

(30) Foreign Application Priority Data
Dec. 30, 2008 (TW) 097151394

(51) Int. Cl. A61K 31/352; A61P 31/14

(52) U.S. Cl. 514/456

(57) ABSTRACT

The invention provides a plant extract composition for treating hepatitis C, including an effective amount of the proanthocyanidins oligomer extracted from a plant material, and a pharmaceutically acceptable carrier or salt, wherein the structure of proanthocyanidins is shown as follows:

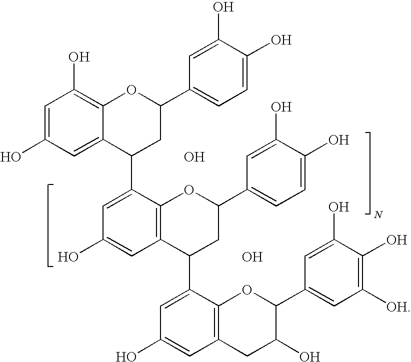

### CROSS REFERENCE TO RELATED APPLICATIONS

This Application claims priority of Taiwan Patent Application No. 097151394, filed on Dec. 30, 2008, the entirety of which is incorporated by reference herein.

### BACKGROUND OF THE INVENTION

1. Field of the Invention

The present invention relates to an anti-hepatitis C virus (HCV) composition, and in particular relates to a composition having proanthocyanidins oligomer extracted from a plant, which is able to inhibit hepatitis C virus activity.

2. Description of the Related Art

About 2-3% of the world population is infected by hepatitis C and the number is increasing by 3-4 million patients every year. Presently, the tested and approved anti-hepatitis C drugs are α-interferon and ribavirin that have been used to enhance the anti-hepatitis C curative effect. However, use of both drugs induces serious side effects and results in drug-resistance.

Also presently, the biochemical and pharmacodynamic activities of the proanthocyanidins have been known to comprise anti-oxidant activity, enzyme inhibition activity, anti-mutation activity, and activity for reducing capillary permeability. Also, the therapeutic effects of the proanthocyanidins are known to comprise anti-inflammation, anti-allergy and anti-ulcer and cancer prophylaxis effe cts, among others.

Taiwan Patent Number I274551 discloses a nutriment containing taurine, β-carotene, proanthocyanidins extracted from grape seeds, vitamin E and vitamin C. The nutriment is found to have an effect on improving chronic hepatitis.

Accordingly, proanthocyanidins is a natural compound isolated from plants, and it has been used to improve human health. However, proanthocyanidins applied to inhibit hepatitis C viral replication has not yet been disclosed.

### BRIEF SUMMARY OF THE INVENTION

The invention provides a plant extract composition for treating hepatitis C, comprising an effective amount of the proanthocyanidins oligomer extracted from a plant material, and a pharmaceutically acceptable carrier or salt.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 shows the structure and mass spectrometry analysis for proanthocyanidins oligomer extracted from grape seeds by the methods used in examples of the invention; and

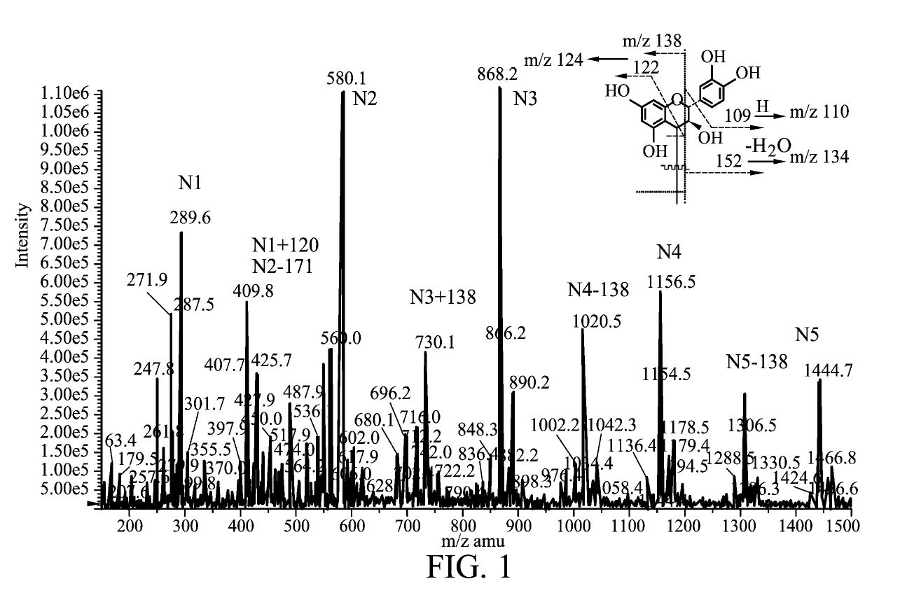

FIG. 2 shows the structure and mass spectrometry analysis for proanthocyanidins oligomer extracted from Polygonum chinense by the methods used in examples of the invention.

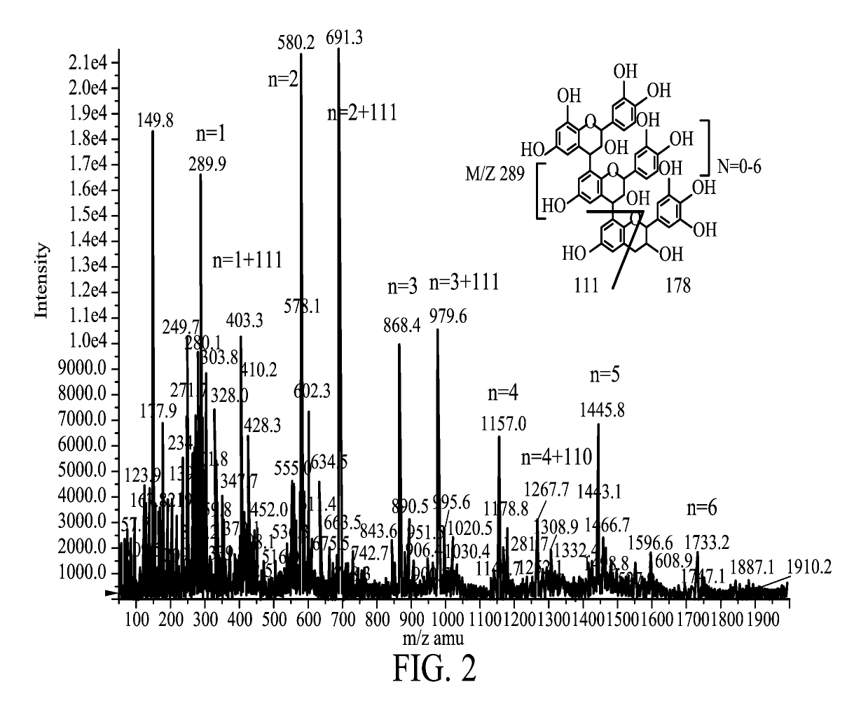

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The invention uses a composition containing proanthocyanidins oligomer extracted from plant as a drug inhibiting hepatitis C and the invention also uses proanthocyanidins oligomer extracted from plant as a nutriment inhibiting the activity of the hepatitis C virus. The hepatitis C replicon huh-luc/noe-ET cell was stably transfected with the I389luc-ubi-NS3-3′/ET gene and resulted in replicative capability of HCV genome. The HCV replication cell is able to express a firefly luciferase-ubiquitin-neomycin fusion protein translated by the internal ribosomal entry site (IRES) of the hepatitis C virus and is able to express the hepatitis C viral nonstructural protein (NS3-5B) including protease, helicase and polymerase translated by the IRES of the encephalomyocarditis virus (EMCV). When the replication complex composed of the IRES of the hepatitis C virus or the nonstructural protein of the hepatitis C virus is influenced by a candidate, the effect of the candidate for inhibiting the activity of the hepatitis C virus repilcon is able to be estimated by determining the intensity of the firefly luciferase activity (Lohmann et al. 1999, Science. 285:110-113). This HCV replicon system has been used worldwide as new drug development tool (Bartenschlager, 2002, Nature reviews in drug discovery. 1: 911-916; Vorlijk et al. 2003, J. Virological Methods. 110: 201-209). The potential candidate to inhibit hepatitis C viral replication can be screened using above described method.

First, a composition containing proanthocyanidins oligomer or proanthocyanidins oligomer is extracted from a plant material. After numerous tests, grape seeds, Polygonum chinense, Ampelopsis brevipedunculata and Ampelopsis cantoniensis were discovered to have the anti-hepatitis C viral replication activity. After tracing of active components of the plant materials, the polar components of the extracts were found to have an abundant amount of the proanthocyanidins oligomer as the active component. Thus, the invention uses extraction technology to extract the parts containing the abundant proanthocyanidins (or the composition having proanthocyanidins) of the plants, to be used as an anti-hepatitis C virus drug.

In the invention, the dried or fresh plants were used as starting materials. The extraction procedure included crashing raw materials, de-esterification, solvent extraction, isolation and purification, concentration, granulation processes, etc. Isolation processes may comprise solvent precipitation, liquid-liquid phase extraction and isolation using resin, etc. In one embodiment, the dried or fresh plant materials may be cut into slices or pulverized and then be extracted with solvents.

The plant materials used for extraction may include grape seeds, Polygonum chinense, Ampelopsis brevipedunculata, Ampelopsis cantoniensis or combinations thereof. The extraction solvent may comprise a polar organic solvent or a mixture of a polar organic solvent and water. The polar organic solvent may comprise acetone, low-alkyl alcohol or ethyl acetate.

In other embodiments, extracted composition obtained from the above described process may be further dissolved in a high polar solvent, and then extracted with a low polar solvent to remove low polar impurities.

The structure of the proanthocyanidins oligomer purified from the process mentioned above is shown in the following, wherein a degree of polymerization of the extracted proanthocyanidins oligomer may be about 1-18.

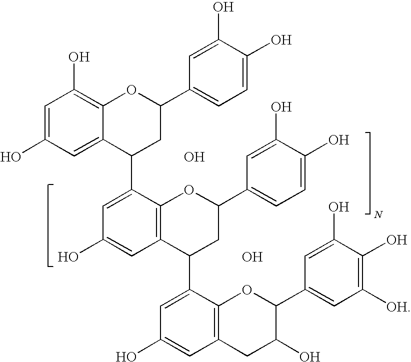

In one embodiment, the extracted plant material may be grape seeds and a degree of polymerization of the proanthocyanidins oligomer extracted therefrom may be about 1-18. In other embodiments, the extracted plant material may be Polygonum chinense and a degree of polymerization of the proanthocyanidins oligomer extracted therefrom may be about 1-18. In another embodiment, the extracted plant material may be Ampelopsis brevipedunculata and a degree of polymerization of the proanthocyanidins oligomer extracted therefrom may be about 1-18. In another embodiment, the extracted plant material may be Ampelopsis cantoniensis and a degree of polymerization of the proanthocyanidins oligomer extracted therefrom may be about 1-18.

The extracted proanthocyanidins oligomer may comprise the proanthocyanidins oligomers with a single degree of polymerization, or the extracted proanthocyanidins oligomer may comprise a mixture of the proanthocyanidins oligomers with different degrees of polymerization.

It was shown that the plant material extract containing proanthocyanidins oligomers or proanthocyanidins oligomers extracted from the plant material inhibited HCV replication over 80% at 50 μg/ml. In one embodiment, the grape seed extract composition containing proanthocyanidins oligomers or proanthocyanidins oligomers extracted from the grape seeds also inhibited HCV replication over 80% at 50 μg/ml. In another embodiment, the Polygonum chinense extract composition containing proanthocyanidins oligomers or proanthocyanidins oligomers extracted from the Polygonum chinense inhibited HCV replication over 80% at 50 μg/ml.

In the invention, the plant material extracted composition containing proanthocyanidins oligomers or proanthocyanidins oligomers may be used to form a pharmaceutical composition for treating hepatitis C. The pharmaceutical composition may comprise the extracted proanthocyanidins oligomers mentioned above and a pharmaceutically acceptable carrier or salt.

The pharmaceutically acceptable carrier may comprise, but is not limited to, a solvent, a dispersion medium, a coating, an antibacterial and antifungal agent, or an isotonic and absorption delaying agent. The pharmaceutical composition can be formulated into dosage forms for different administration routes utilizing conventional methods.

The pharmaceutically acceptable salt may comprise, but is not limited to, inorganic cation salts including alkali metal salts such as sodium salt, potassium salt or amine salt, alkaline-earth metal salt such as magnesium salt or calcium salt, the salt containing bivalent or quadrivalent cation such as zinc salt, aluminum salt or zirconium salt. In addition, the pharmaceutically acceptable salt may also comprise organic salt including dicyclohexylamine salt, methyl-D-glucamine, and amino acid salt such as arginine, lysine, histidine, or glutamine.

The pharmaceutical composition may be administered orally, parentally by an inhalation spray or via an implanted reservoir. The parental method may comprise subcutaneous, intracutaneous, intravenous, intramuscular, intraarticular, intraarterial, intrasynovial, intrasternal, intrathecal, and intraleaional, as well as infusion techniques.

An oral composition can comprise, but is not limited to, tablets, capsules, emulsions and aqueous suspensions, dispersions and solutions.

### EXAMPLE

### Example 1

1 Kg of the dried Polygonum chinense root was dipped in 95% ethanol and shaked by 120 rpm for 3 days at room temperature to extract active components. After the extracted solution was filtered, the extract was concentrated in vacuo to a minimum. 90 g of the crude extract was dissolved/suspended into a mixture of water and ethanol (95:5) and then the mixture was sequentially partitioned with hexane, ethyl ether and ethyl acetate to afford hexane layer, ethyl ether layer, ethyl acetate layer, and residue, respectively. The extraction process is shown in the following:

The extract from Polygonum chinense root was mixed well with hexane, placed in a funnel and left standing for layer separation. Following layer separation, the upper layer of the mixed solution was obtained. The process was repeated for three times and the hexane layer was collected. Next, the lower layer (water layer) was mixed well with ethyl ether and placed in a funnel and left standing for layer separation. Following layer separation, the upper layer of the solution was obtained. The process was repeated for three times and an ethyl ether layer was collected. After that, the lower layer (water layer) was mixed well with ethyl acetate and placed in a funnel and left standing for layer separation. Following layer separation, the upper layer of the solution was obtained. The process was repeated for three times and an ethyl acetate layer and the water layer from the third process were obtained. Each layered solution were dried in vacuo to afford 4.6 g of hexane layer extract, 2.9 g of ethyl ether layer extract, 5.6 g of ethyl acetate layer extract and 60-70 g of water layer extract, respectively.

Four extracts were performed to determine inhibiting activity of hepatitis C virus. The test results showed that the water layer extract had the highest inhibiting activity of Hepatitis C virus. The cytotoxicity CC₅₀ thereof was greater than 1000 μg/ml and inhibition activity IC₅₀ thereof was 5.2±1.2 μg/ml. The inhibition activity IC₅₀ of the ethanol crude extract was 11.82±3.3 μg/ml and further extraction of the ethanol crude extract resulted in increasing 2 fold inhibition activity compared to ethanol crude extract.

TABLE 1
Inhibition test of hepatitis C viral replication activity using
extracts which was extracted following with hexane, ethyl ether,
ethyl acetate, water, and ethanol from the roots of Polygonum
chinense, respectively. The original ethanol crude extract and
final water layer extract were used to test anti-HCV activity in
huh-luc/noe-ET cells.

| Sample | IC₅₀ (μg/ml) |
|---|---|
| ethanol crude extract | 11.8 ± 3.3 |
| Water layer extract | 5.2 ± 1.2 |

Table 1 showed that the water extract had the best Hepatitis C virus inhibition activity. Therefore, the water extract was selected for further purification using open-column chromatography.

Open Column Chromatography:

1.0077 g of the water layer extract was separated by open column chromatography (column packed with RP C-18/30.4419 g of silica; 2.2×25.3 cm) with successively changes of the mobile phase comprising a mixture of water and acetone (500 mL, water:acetone=4:1), a mixture of water and acetone (1,000 mL, water:acetone=3:1), a mixture of water and acetone (2:1), a mixture of water and acetone (1:1) and acetone. The eluents were analyzed using thin layer chromatography and combined the same constitute to obtain 10 subfractions to perform the inhibition test of hepatitis C virus. Table 2 shows the results of 6 of the 10 samples, wherein the extract from the mixture of water and acetone (3:1, fraction 126-250 ml) have inhibition activity of hepatitis C virus.

TABLE 2
Inhibition test of hepatitis C viral replication activity using the
crude and further fractionated extracts of Polygonum chinense.

| | Activity (50 μg/ml) | Cytotoxicity |
|---|---|---|
| Sample | Inhibition (%) | CC₅₀ (μg/ml) |
| Polygonum chinense | 39.6 ± 4.7 | 900.1 |
| water layer extract | | |
| Polygonum chinense | 43.9 ± 5.4 | >1000 |
| water:acetone = 4:1 | | |
| 1-125 ml | | |
| Polygonum chinense | 37.1 ± 5.0 | >1000 |
| water:acetone = 4:1 | | |
| 126-250 ml | | |
| Polygonum chinense | 32.7 ± 8.0 | 757.5 |
| water:acetone = 4:1 | | |
| 251-500 ml | | |
| Polygonum chinense | 40.6 ± 16.3 | 413.8 |
| water:acetone = 3:1 | | |
| 1-125 ml | | |
| Polygonum chinense | 72.3 ± 4.4 | 269.1 |
| water:acetone = 3:1 | | |
| 126-250 ml | | |
| Polygonum chinense | 13.7 ± 14.8 | >1000 |
| water:acetone = 3:1 | | |
| 251-500 ml | | |

### Example 2

1. 5 g of the dried Polygonum chinense root was dipped in 50 ml of pure water and shaked at 120 rpm for 24 hours at room temperature to produce an extract solution. The extracts were concentrated in vacuo and 0.0676 g of the extract was used to test the inhibition activity of hepatitis C virus. The data showed that this extract can inhibit 58.7±5.9% of hepatitis C viral replication at concentration of 50 μg/ml.

2. The same process as step 1 was performed, wherein the solvent was changed to be 50 ml of acetone. 0.0043 g of the extract was used to test the inhibition activity of hepatitis C virus. The data showed that this extract can inhibit 88.7±1.3% of hepatitis C viral replication at concentration of 50 μg/ml.

3. The same process as step 1 was performed, wherein the solvent was changed to be 50 ml of a mixture of acetone and pure water (1:1). 0.0488 g of the extract was used to test the inhibition activity of hepatitis C virus. The data showed that this extract can inhibit 87.3±1.9 of hepatitis C viral replication at concentration of 50 μg/ml.

4. The same process as step 1 was performed, wherein the solvent was changed to be 50 ml of a mixture of acetone and pure water (1:2). 0.0082 g of the extract was used to test the inhibition activity of hepatitis C virus. The data showed that this extract can inhibit 79.9±2.2% of hepatitis C viral replication at concentration of 50 μg/ml.

5. The same process as step 1 was performed, wherein the solvent was changed to be 50 ml of a mixture of acetone and pure water (2:1). 0.0522 g of the extract was used to test the inhibition activity of hepatitis C virus. The data showed that this extract can inhibit 82.3±2.7% of hepatitis C viral replication at concentration of 50 μg/ml.

6. The same process as step 1 was performed, wherein the solvent was changed to be 50 ml of methanol. 0.0379 g of the extract was used to perform the hepatitis C virus activity inhibition test. For a 50 μg/ml concentration of the extract, the inhibition rate to the hepatitis C virus was 83.5±2.8%.

7. The same process as step 1 was performed, wherein the solvent was changed to be 50 ml of a mixture of methanol and pure water (1:1). 0.0435 g of the extract was used to test the inhibition activity of hepatitis C virus. The data showed that this extract can inhibit 84.0±4.6% of hepatitis C viral replication at concentration of 50 μg/ml.

8. The same process as step 1 was performed, wherein the solvent was changed to be 50 ml of a mixture of methanol and pure water (1:2). 0.0622 g of the extract was used to test the inhibition activity of hepatitis C virus. The data showed that this extract can inhibit 79.0±6.8% of hepatitis C viral replication at concentration of 50 μg/ml.

9. The same process as step 1 was performed, wherein the solvent was changed to be 50 ml of a mixture of methanol and water (2:1). 0.0272 g of the extract was used to test the inhibition activity of hepatitis C virus. The data showed that this extract can inhibit 85.0±3.4% of hepatitis C viral replication at concentration of 50 μg/ml.

10. The same process as step 1 was performed, wherein the solvent was changed to be 50 ml of ethyl acetate-saturated pure water solution. 0.0337 g of the extract was used to test the inhibition activity of hepatitis C virus. The data showed that this extract can inhibit 75.7±0.8% of hepatitis C viral replication at concentration of 50 μg/ml.

The inhibiting activities of hepatitis C virus test by using the Polygonum chinense root extracted with the solvents mentioned above are shown in Table 3.

TABLE 3
Inhibition test of hepatitis C viral replication activity using the root
of Polygonum chinense, following crude and fractional extraction
with various solvents and ratios.

| | Activity | |
|---|---|---|
| Root of | (50 μg/ml) | Cytotoxicity |
| Polygonum chinense | Inhibition (%) | CC₅₀ (μg/ml) |
| water | 58.7 ± 5.9 | >333 |
| acetone | 88.7 ± 1.3 | >333 |
| acetone/water = 1:1 | 87.3 ± 1.9 | >333 |
| acetone/water = 1:2 | 79.9 ± 2.2 | >333 |
| acetone/water = 2:1 | 82.3 ± 2.7 | >333 |
| methonal | 83.5 ± 2.8 | >333 |
| methonal/water = 1:1 | 84.0 ± 4.6 | >333 |
| methonal/water = 1:2 | 79.0 ± 6.8 | >333 |
| methonal/water = 2:1 | 85.0 ± 3.4 | >333 |
| ethyl acetate-saturated | 75.7 ± 0.8 | >333 |
| pure water | | |

### Example 3

1. 5 g of the grape seeds (normal grape) was dipped in 50 ml of pure water and shaked at 120 rpm for 24 hours at room temperature to produce extract solution. The extracts were concentrated in vacuo and 0.236 g of the extract was used to test the inhibition activity of hepatitis C virus. The inhibition rate of the hepatitis C virus was 81.6±3.3% at concentration of 50 μg/ml.

2. The same process as step 1 was performed, wherein the solvent was changed to be 50 ml of acetone. 0.687 g of the extract was used to test the inhibition activity of hepatitis C virus. The inhibition rate of the hepatitis C virus was 5.0±12.2% at concentration of 50 μg/ml.

3. The same process as step 1 was performed, wherein the solvent was changed to be 50 ml of a mixture of acetone and pure water (1:1). 0.1164 g of the extract was used to test the inhibition activity of hepatitis C virus. The inhibition rate of the hepatitis C virus was 82.1±4.7% at concentration of 50 μg/ml.

4. The same process as step 1 was performed, wherein the solvent was changed to be 50 ml of a mixture of acetone and pure water (1:2). 0.034 g of the extract was used to test the inhibition activity of hepatitis C virus. The inhibition rate of the hepatitis C virus was 87.3±2.9% at concentration of 50 μg/ml.

5. The same process as step 1 was performed, wherein the solvent was changed to be 50 ml of a mixture of acetone and pure water (2:1). 0.1213 g of the extract was used to test the inhibition activity of hepatitis C virus. The inhibition rate of the hepatitis C virus was 80.4±7.9% at concentration of 50 μg/ml.

6. The same process as step 1 was performed, wherein the solvent was changed to be 50 ml of saturated ethyl acetate solution. 0.0506 g of the extract was used to test the inhibition activity of hepatitis C virus. The inhibition rate of the hepatitis C virus was 87.1±2.5% at concentration of 50 μg/ml.

The inhibition activity of hepatitis C virus test by using rape seeds extracted with the solvents mentioned above are shown in Table 4.

TABLE 4
Inhibition test of hepatitis C viral replication activity using the
crude and fractionated extracts of grape seeds.

| | Activity | |
|---|---|---|
| Grape seeds | (50 μg/ml) | Cytotoxicity |
| (normal grape) | Inhibition (%) | CC₅₀ (μg/ml) |
| water | 81.6 ± 3.3 | >333 |
| acetone | 5.0 ± 12.2 | >333 |
| acetone/water = 1:1 | 82.1 ± 4.7 | >333 |
| acetone/water = 1:2 | 87.3 ± 2.9 | >333 |
| acetone/water = 2:1 | 80.4 ± 7.9 | >333 |
| ethyl acetate-saturated | 87.1 ± 2.5 | >333 |
| pure water | | |

### Example 4

1. 5 g of the flesh of the Ampelopsis brevipedunculata was dipped in 50 ml of water and shaked at 120 rpm for 24 hours at room temperature to produce an extract solution. The extracts were concentrated in vacuo and 0.0175 g of the extract was used to test the inhibition activity of hepatitis C virus. The inhibition rate of the hepatitis C virus was 84.7±1.7% at concentration of 50 μg/ml.

2. The same process as step 1 was performed, wherein the solvent was changed to be 50 ml of methanol. 0.0474 g of the extract was used to test the inhibition activity of hepatitis C virus. The inhibition rate of the hepatitis C virus was 92.1±0.7% at concentration of 50 μg/ml.

3. The same process as step 1 was performed, wherein the solvent was changed to be 50 ml of a mixture of methanol and pure water (1:1). 0.0279 g of the extract was used to test the inhibition activity of hepatitis C virus. The inhibition rate of the hepatitis C virus was 92.1±1.2% at concentration of 50 μg/ml.

4. The same process as step 1 was performed, wherein the solvent was changed to be 50 ml of a mixture of methanol and pure water (1:2). 0.0674 g of the extract was used to test the inhibition activity of hepatitis C virus. The inhibition rate of the hepatitis C virus was 92.7±0.3% at concentration of 50 μg/ml.

5. The same process as step 1 was performed, wherein the solvent was changed to be 50 ml of a mixture of methanol and pure water (2:1). 0.0536 g of the extract was used to test the inhibition activity of hepatitis C virus. The inhibition rate of the hepatitis C virus was 90.0±2.5% at concentration of 50 μg/ml.

6. The same process as step 1 was performed, wherein the solvent was changed to be 50 ml of ethyl acetate-saturated pure water solution. 0.0408 g of the extract was used to test the inhibition activity of hepatitis C virus. The inhibition rate of the hepatitis C virus was 91.3±0.7% at concentration of 50 μg/ml.

The inhibition activity of hepatitis C virus test by using grape seeds extracted with the solvents mentioned above are shown in Table 5.

TABLE 5
Inhibition test of hepatitis C viral replication activity using the
crude and fractionated extracts of Ampelopsis brevipedunculata.

| | Activity (50 μg/ml) | Cytotoxicity |
|---|---|---|
| Ampelopsis brevipedunculata | Inhibition (%) | CC₅₀ (μg/ml) |
| water | 84.7 ± 1.7 | >1000 |
| methanol | 92.1 ± 0.7 | >1000 |
| methanol/water = 1:1 | 92.1 ± 1.2 | >1000 |
| methanol/water = 1:2 | 92.7 ± 0.3 | >1000 |
| methanol/water = 2:1 | 90.0 ± 2.5 | >1000 |
| ethyl acetate-saturated pure water | 91.3 ± 0.7 | >1000 |

### Example 5

100 g of the dried Ampelopsis cantoniensis was dipped in 95% ethanol and shaked at 120 rpm for 3 days at room temperature to produce extract solution. The extract solution was concentrated in vacuo and used to test the inhibition activity of hepatitis C virus. The inhibition rate of the hepatitis C virus was 72-80% at concentration of 50 μg/ml.

While the invention has been described by way of example and in terms of the preferred embodiments, it is to be understood that the invention is not limited to the disclosed embodiments. In contrast, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A plant extract composition for treating hepatitis C, comprising:
an effective amount of the proanthocyanidins oligomer extracted from a plant material; and
a pharmaceutically acceptable carrier or salt, wherein the structure of proanthocyanidins is shown as follows:

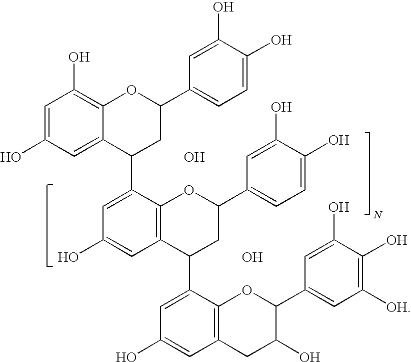

2. The plant extract composition for treating hepatitis C as claimed in claim 1, wherein a degree of polymerization of the proanthocyanidins oligomer is about 1-18.

3. The plant extract composition for treating hepatitis C as claimed in claim 1, wherein the proanthocyanidins oligomer comprises the proanthocyanidins oligomers with a single degree of polymerization or a mixture of the proanthocyanidins oligomers with different degrees of polymerization.

4. The plant extract composition for treating hepatitis C as claimed in claim 1, wherein the plant material comprises grape seeds, Polygonum chinense, Ampelopsis brevipedunculata, Ampelopsis cantoniensis or combinations thereof.

5. The plant extract composition for treating hepatitis C as claimed in claim 1, wherein the plant material comprises grape seeds.

6. The plant extract composition for treating hepatitis C as claimed in claim 5, wherein a degree of polymerization of the proanthocyanidins oligomer is about 1-18.

7. The plant extract composition for treating hepatitis C as claimed in claim 1, wherein the plant material comprises Polygonum chinense.

8. The plant extract composition for treating hepatitis C as claimed in claim 7, wherein a degree of polymerization of the proanthocyanidins oligomer is about 1-18.

9. The plant extract composition for treating hepatitis C as claimed in claim 1, wherein the plant material comprises Ampelopsis brevipedunculata.

10. The plant extract composition for treating hepatitis C as claimed in claim 9, wherein a degree of polymerization of the proanthocyanidins oligomer is about 1-18.

11. The plant extract composition for treating hepatitis C as claimed in claim 1, wherein the plant material comprises Ampelopsis cantoniensis.

12. The plant extract composition for treating hepatitis C as claimed in claim 11, wherein a degree of polymerization of the proanthocyanidins oligomer is about 1-18.

13. The plant extract composition for treating hepatitis C as claimed in claim 1, wherein a solvent used to extract the plant material comprises a polar organic solvent or a mixture of a polar organic solvent and water.

14. The plant extract composition for treating hepatitis C as claimed in claim 1, wherein a solvent used to extract the plant material comprises acetone or a mixture of acetone and water.

15. The plant extract composition for treating hepatitis C as claimed in claim 1, wherein a solvent used to extract the plant material comprises low-alkyl alcohol or a mixture of low-alkyl alcohol and water.

16. The plant extract composition for treating hepatitis C as claimed in claim 1, wherein a solvent used to extract the plant material comprises ethyl acetate or a mixture of ethyl acetate and water.
